SEQUENCE LISTING (7 sequences)

SEQ ID NO: 1 - DNA, 350 bp, Artificial Sequence
  synthesized
caggtgtctcatttaagtcaacctcccttaagtttccacttgaaccttttgatgcacagaaaccagaaaactcagatgttgtctcagtattggcaatgtccattgctagcttggcttcagtagggaagagtaaccgtgcaaatgccactctattttctaggtacagcagcttcatgtgcaaatcatcagataaagtgtgggaggtgataaaggcatcaccagccatcggattcctcctcatttctccctccagaacatcaatgcatatacgatctttatttgtttcttttccctgctctgtttttgcctgataatctttcggccttgtcaatcttctacagatgctgacg

SEQ ID NO: 2 - DNA, 20 bp, Artificial Sequence
  synthesized
tgatgagaacactgcagttg

SEQ ID NO: 3 - DNA, 20 bp, Artificial Sequence
  synthesized
caagagacttcgtcctagga

SEQ ID NO: 4 - DNA, 1113 bp, Artificial Sequence
  synthesized
ggctaattcgattgaaccgtcatcatctttgagctttacttcgtcttctcatctgtcaaatggctcaattagtcataaccaatcttctttttcggctcctgagaagggggttaatcttgaagtttttagtctaaataagcttagttctagtttggaacaactcctgattgattccacttgtgaatatagtgatgctgaaataattgttgagggtatcccagtttgtgttcatcgatgtattttggctgcaaggagtaagtttttttatgagttatttaagcgtgagaaggggtcagttgataaggagggaaaaccaaagtatcccatgagtgagttgttgccgtatggcaaggttggatatgaagcctttttaattttcttaagttacacgtattccggagttggtttcacttttccaggtcaggatgccttctttattctggttacaatttacttttcataagcaaatttacctacttaatcaatacatgcctttggtatagagcttgggaactgccttctggatcaatctgtttttaatgcaaactgaaaagatgaatacaataaagtgattttttttttcaagtacaataataagatcctctgtgtgtttttgttcattatccacatctgtataatttcaatttgcagacaccgagaggaacatatgttgacttaataatctacgtatttgcagcgacgtctacttaattttgtgaatacgtgtaacttaagaaaattaaaaaggcttcatatccaaccttgccatacggcaacaactcactcatgggatactttggttttccctccttatcaactgaccccttctcacgcttaaataactcataaaaaaacttactccttgcagccaaaatacatcgatgaacacaaactgggataccctcaacaattatttcagcatcactatattcacaagtggaatcaatcaggagttgttccaaactagaactaagcttatttagactaaaaacttcaagattaacccccttctcaggagccgaaaaagaagattggttatgactaattgagccatttgacagatgagaagacgaagtaaagctcaaagatgatgacggttcaatcgaattagcc

SEQ ID NO: 5 - DNA, 21 bp, Artificial Sequence
  synthesized
tacacaaactgggatacgctc

SEQ ID NO: 6 - DNA, 21 bp, Artificial Sequence
  synthesized
tgttatgactaattgagccag

SEQ ID NO: 7 - DNA, 21 bp, Artificial Sequence
  synthesized
tggttatgactaattgcgcca

(19) United States
(12) Patent Application Publication
(10) Pub. No.: US 2025/0304991 A1
(43) Pub. Date: Oct. 2, 2025

(54) CREATE HUANGLONGBING TOLERANCE BY SILENCING A CITRUS NEGATIVE IMMUNE REGULATOR

(71) Applicant: UNIVERSITY OF FLORIDA RESEARCH FOUNDATION, INCORPORATED, Gainesville, FL (US)

(72) Inventors: Choaa Amine A. EL-MOHTAR, Gainesville, FL (US); Xudong ZHANG, Gainesville, FL (US); Cecile J. ROBERTSON, Gainesville, FL (US); William O. DAWSON, Gainesville, FL (US); Zhonglin MOU, Gainesville, FL (US)

(21) Appl. No.: 18/268,280

(22) PCT Filed: Dec. 17, 2021
(86) PCT No.: PCT/US2021/063987

§ 371 Date: Jun. 19, 2023

Related U.S. Application Data
(60) Provisional application No. 63/127,523, filed on Dec. 18, 2020.

(51) Int. Cl. C12N 15/82; C12N 9/22; C12N 15/11

(52) U.S. Cl. CPC C12N 15/8281; C12N 9/226; C12N 15/111; C12N 15/8203; C12N 15/8213; C12N 2310/20

(57) ABSTRACT

Disclosed are methods for inducing tolerance to Huanglongbing (HLB) in citrus plants. It was found that silencing negative regulators of the plant immune system will provide resistance or tolerance to diseases, particularly HLB. NPR3 is silenced using citrus tristeza virus-based plant mediated RNA interference (CTV-RNAi) to create citrus plants that are resistance to HLB. Also disclosed are CTV-RNAi vectors designed to silence negative regulators of the plant immune system.

### BACKGROUND

Citrus is the major agricultural crop in Florida, valued at $10 billion and provides 76,000 jobs. For many rural communities, citrus is the economic base of the local economy. California is second to Florida in total citrus acreage, but has a larger share of the higher value, fresh market produce. Huanglongbing (HLB), also called citrus greening, is one of the most devastating diseases of citrus worldwide. It is caused by unculturable phloem-limited bacteria that belong to the Candidatus Liberibacter genus including Ca. L. asiaticus (CLas), Ca. L. africanus, and Ca. L. americanus. The bacterial pathogens are transmitted by the Asian citrus psyllid Diaphorina citri or the African citrus psyllid Trioza erytreae.

In Florida, HLB is a disaster for the citrus industry. HLB has spread to almost all trees, reducing yields and the quality of juice. Since HLB was first found in 2005, total citrus acreage and yield have shrunk 43% and 75%, respectively (FL DACS, 2020). Production of sweet orange dropped from 150 million boxes in 2005-2006 to 72 million boxes in 2018-2019, causing the closure of more than 75% of the citrus packinghouses (Graham et al., 2020). HLB is in Texas and is spreading. In California, the frequency of detection of HLB in residential trees is increasing (Kumagai et al., 2013; McRoberts et al., 2019), and the vector, the Asian citrus psyllid, continues to spread into commercial areas. Without new solutions, these citrus industries may follow a pattern similar to that in Florida.

Although several therapeutic treatments including antibiotics and thermotherapy that purportedly kill CLas in the phloem have been tested, they are costly and do not substantially reverse the health of HLB trees (Blaustein et al., 2017; Graham and Morgan, 2017). In this embodiment, a novel strategy that can be used as both therapeutic and preventative measures for controlling HLB is described.

### BRIEF DESCRIPTION OF THE DRAWINGS

FIG. 1 shows the AtNPR1 (Arabidopsis thaliana NPR1) protein levels in the three HLB-tolerant lines that were detected with a specific anti-AtNPR1 antibody. The Ponceau S staining of Rubisco was used as the loading control.

FIG. 2A-G are representative HLB-tolerant transgenic line 13-29. (A) The original plant of the transgenic line ‘Hamlin’ 13-29, which was produced in 2008, has been CLas positive (indicated by the Ct value) for eight years and has continued growing normally. (B) to (G) Six progeny plants of the transgenic line. These plants are also CLas positive (indicated by the Ct values) but do not have HLB symptoms.

FIG. 3 is a diagram of CTV vector strategies.

FIG. 4 shows the newly developed citrus NPR1 antibody. The AtNPR1 and citrus NPR1 proteins were transiently expressed in Nicotiana benthamiana leaves as previously described (Wang et al., 2015). Total protein was extracted, separated by SDS-PAGE, transferred onto a nitrocellulose membrane, and probed with the citrus NPR1 antibody. The empty T-DNA vector and AtNPR1 were included as the controls. The asterisk shows a nonspecific band present in all three samples, indicating equal loading.

FIG. 5 is a western blot quantifying citrus NPR1 protein levels in several RNAi lines. About 100 mg bark tissues were collected from each RNAi line. Total protein was extracted, separated by SDS-PAGE, transferred onto a nitrocellulose membrane, and probed with the citrus NPR1 antibody. Two independent lines for each construct were analyzed. Plants without treatment or treated with empty CTV vector were included as the controls. The asterisks show a nonspecific band present in all samples, indicating equal loading.

FIG. 6 is a Huanglongbing (HLB) test result of control plants (left) and citrus NPR3 (CsNPR3) RNAi lines (right). The control plants were infected with the CTV empty vector. CTV and CTV-RNAi constructs were graft-transmitted into sweet orange ‘Pineapple’ seedlings. After systemic CTV infection was confirmed with ELISA, the citrus trees were inoculated in a containment growth room with CLas-infected psyllids for 2-4 weeks. The plants were all successfully infested. The control plants carrying the CTV empty vector exhibited severe HLB symptoms and died quickly. In contrast, the CsNPR3 RNAi lines showed no or very mild HLB symptoms. Importantly, progenies propagated by grafting from the original CsNPR3 RNAi plants displayed no HLB symptoms. These results indicate that CsNPR3 RNAi provided robust tolerance to the HLB disease.

FIG. 7 is a quantitative PCR assay measuring CsNPR3 relative expression levels in the RNAi lines. About 100 mg bark tissues for each sample were collected from the RNAi lines and the control plants. Total RNA was extracted and analyzed for expression of the CsNPR3 gene using quantitative real-time PCR. Expression was normalized against the constitutively expressed CsGAPDH gene. Values are means±standard deviation of three independent samples. Different letters above the bars indicate significant differences (P<0.05, Student's t test). This result indicates that the expression levels of the CsNPR3 gene in the RNAi lines were significantly reduced.

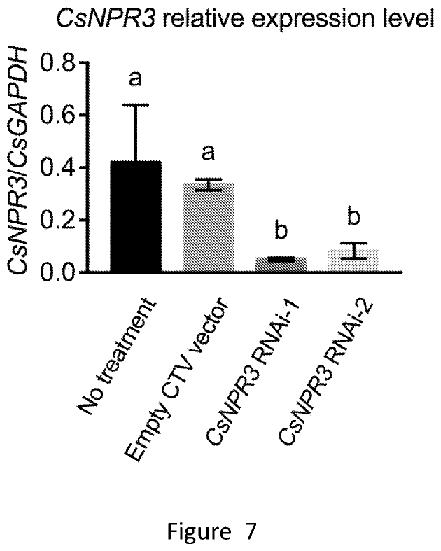

### REFERENCE TO ELECTRONIC SEQUENCE LISTING

The application contains a Sequence Listing which has been submitted electronically in ASCII format and is hereby incorporated by reference in its entirety. Said ASCII copy, created on Jan. 8, 2024, is named “10457495US1_ST25.txt” and is 3,362 bytes in size. The sequence listing contained in this.txt file is part of the specification and is hereby incorporated by reference herein in its entirety.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

Practice of the methods, as well as preparation and use of the compositions disclosed herein employ, unless otherwise indicated, conventional techniques in molecular biology, biochemistry, DNA and RNA structure and analysis, computational chemistry, cell culture, recombinant DNA and related fields as are within the skill of the art. These techniques are fully explained in the literature. See, e.g., Sambrook et al. MOLECULAR CLONING: A LABORATORY MANUAL, 2d ed., Cold Spring Harbor Laboratory Press, 1989; 3d ed., 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego.

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

The term “alter(ing) DNA sequence, gene structure and function” refers to 1) introducing a change (deletion or insertion, i.e. indel) of deoxynucleotide(s) in the gene's coding sequence (CDS), 2) introducing a change (indel) of deoxynculeotide(s) in the promoter region, or 3) introducing a change (indel) of deoxynculeotide(s) in the gene's 5′ untranslated region (UTR). Such changes in the gene's coding sequence are expected to result in 1) a change (deletion or insertion) of amino acid(s) in the gene product encoded by the gene, and/or 2) a frameshift of the open reading frame of the gene leading to a pre-matured polypeptide, non-functional polypeptide or a function-impacted polypeptide.

The term “disrupt(s)” or “disrupting” with respect to a gene refers to a decrease or elimination of expression of a gene product encoded by a gene or to change the gene's normal function.

The term “citrus” refers to any known variety, cultivar, breeding line or accession of plants in the genus Citrus and sexually compatible genera Fortunella and Poncirus. In some recent classification, plants of Fortunella and Poncirus have been placed in the genus Citrus.

Citrus varieties contemplated by this disclosure include, but are not limited to, cultivated citrus types such as sweet oranges, blood oranges, navel oranges, round oranges, grapefruit, pomelo or pumello, citrons, lemons, limes, mandarins, Clementine mandarins, Satsuma mandarins, tangerines, tangors, tangelos, trifoliate oranges, citranges, citrumelos, or the like.

The terms “CsNPR3” or “Citrus Non-expresser of Pathogenesis Related Genes 3” and “CsNPR4” or “Citrus Non-expresser of Pathogenesis Related Genes 4” refer to members of a large group of negative regulators of the plant immune system. NPR3 and NPR4 are salicylic acid (SA) receptors that function as adaptors of a ubiquitin E3 ligase to mediate NPR1degradations. In unhealthy plant cells, SA levels are high, NPR3/4 are bound to SA and cannot repress NPR1, which leads to activation of the plant immune system. This defense response has been shown in Arabidopsis. CsNPR1 (Cs4g14600) is the functional NPR1 in citrus plants. CsNPR3 (Cs2g10790) and CsNPR4 (Cs7g18600) are the closest homologs of NPR3/4 in citrus. The CsNPR1 protein may be similarly regulated by CsNPR3 and CsNPR4 in regulating systemic acquired resistance (SAR) in citrus plants.

The term “Citrus tristeza virus (CTV)” refers to a member of the genus Closterovirus of the family Closteroviridae, the largest and the most complex plant viral family. Single-stranded RNA genome of ˜19.3 kb is encapsidated by two coat proteins (CP) making a long flexuous virions (2000 nm by 10-12 nm). CTV vector is a gene delivering system that has been shown to be an efficient expression vector capable of expressing more than one foreign gene engineered at different positions in its genome either as extra gene or substitution of some non-essential genes using homologous and heterologous sub-genomic RNA (sgRNA) controller elements.

The term “Huanglongbing (HLB)” refers to the disease citrus greening, is one of the most destructive diseases of citrus worldwide. It is caused by unculturable phloem-limited bacteria that belong to the Candidatus Liberibacter genus including Ca. L. asiaticus (CLas), Ca. L. africanus, and Ca. L. americanus. The bacterial pathogens are transmitted by the Asian citrus psyllid Diaphorina citri or the African citrus psyllid Trioza erytreae.

The term “operably linked” refers to sequences joined as part of the same nucleic acid molecule, suitably positioned and oriented for transcription to be initiated from the promoter. DNA operably linked to a promoter is under transcriptional initiation regulation of the promoter or in functional combination therewith.

The term “RNA interference (RNAi)” refers to a process by which double-stranded RNA (dsRNA) is used to silence gene expression. While not wanting to be bound by theory, RNAi begins with the cleavage of longer dsRNAs into small interfering RNAs (siRNAs) by an RNase III-like enzyme, dicer. SiRNAs are dsRNAs that are usually about 19 to 28 nucleotides, or 20 to 25 nucleotides, or 21 to 22 nucleotides in length and often contain 2-nucleotide 3′ overhangs, and 5′ phosphate and 3′ hydroxyl termini. One strand of the siRNA is incorporated into a ribonucleoprotein complex known as the RNA-induced silencing complex (RISC). RISC uses this siRNA strand to identify mRNA molecules that are at least partially complementary to the incorporated siRNA strand, and then cleaves these target mRNAs or inhibits their translation. Therefore, the siRNA strand that is incorporated into RISC is known as the guide strand or the antisense strand. The other siRNA strand, known as the passenger strand or the sense strand, is eliminated from the siRNA and is at least partially homologous to the target mRNA. Those of skill in the art will recognize that, in principle, either strand of an siRNA can be incorporated into RISC and function as a guide strand. However, siRNA design (e.g., decreased siRNA duplex stability at the 5′ end of the desired guide strand) can favor incorporation of the desired guide strand into RISC.

The terms “RNA interfering inducer” or “RNAi inducer” refer to RNA molecules that can be used in RNAi. RNAi inducers can be but are not limited to dsRNA, shRNA, siRNA, amiRNA, and miRNA. DsRNA are the most commonly used RNAi inducers. RNAi inducer sequences can be cloned from cDNA or synthetically designed by prediction software to increase specificity. The RNAi inducer can silence the expression of a gene product. The term “silence” or “silencing” as used herein refers to partial or complete reduction in expression of the gene product.

The term “sequence” refers to a nucleotide sequence of any length, which can be DNA or RNA; can be linear, circular or branched and can be either single-stranded or double stranded.

The term “systemic acquired resistance (SAR)” refers to plant immunity, to a broad spectrum of pathogens that is normally established after a primary exposure to avirulent pathogens. Systemic acquired resistance (SAR) is a defense response that can be triggered by a local hypersensitive response (HR) to an avirulent pathogen, which renders uninfected parts of the plant resistant to a variety of normally virulent pathogens. SAR is thought to be the consequence of the concerted activation of many genes that are often referred to as pathogenesis-related (PR) gene.

The term “tolerance” refers to the capacity to endure continued subjection to something, especially a disease, antigen, or environmental conditions, without adverse reaction. Tolerance to disease is determined in plants when after numerous rounds of inoculation the plants continue to grow normally and only exhibit mild symptoms.

### DETAILED DESCRIPTION

It was found in transgenic citrus plants that high levels of the NPR1 protein was correlated with HLB tolerance or resistance. Citrus trees genetically modified to overexpress Arabidopsis NPR1 (AtNPR1) confirmed that NPR1 can create tolerance to HLB. However, these trees are genetically modified (GM) and will have to go through the approval process that cannot be accomplished in time to save the Florida citrus industry. A panel of negative immune regulators were knocked down in Citrus macrophylla plants utilizing CTV-RNAi constructs.

Knocking down CsNPR3 (Cs2g10790) was shown to increase citrus NPR1 protein levels. Based on these findings it was determined that silencing CsNPR3 expression citrus trees can gain tolerance to HLB.

In certain embodiments, provided is a method for activating systemic acquired resistance (SAR) in a citrus plant. The method involves introducing into a citrus plant cell or tissue of the citrus plant a Citrus tristeza virus (CTV) viral vector engineered to produce an RNA interfering (RNAi) inducer targeting a negative immune regulator CsNPR3 (Cs2g10790), wherein the inducer disrupts CsNPR3 such that SAR is activated in the citrus plant.

In a further embodiment, provided is a method for activating systemic acquired resistance (SAR) in a citrus plant that involves introducing into citrus plant cells a non-naturally occurring gene editing system. The gene editing system includes a (i) nucleic acid sequence encoding a nuclease comprising a Clustered Regularly Interspaced Short Palindromic Repeats (CRISPR)-associated (Cas) protein and (ii) a nucleic acid sequence encoding gRNA sequence that targets CsNPR3 (Cs2g10790). The gene editing system is engineered to alter CsNPR3, the alteration, for example, can disrupt expression and/or function of CsRPR3. The sequences (i) and/or (ii) can be operably linked to a regulatory element functional in a plant cell.

In a specific embodiment described herein, provided is a method for ameliorating HLB in citrus trees by decreasing expression of negative regulators of the citrus immune system. One embodiment described within is a method for the creation of HLB tolerance in citrus by silencing CsNPR3 (Cs2g10790) using citrus triseza virus-based plant mediated RNA interference constructs. The constructs are introduced into a bark flap inoculated into Citrus macrophylla plants. In one embodiment, CRISPR/Cas9 is used to knockout CsNPR3 (Cs2g10790) to produce non-genetically modified HLB tolerance citrus trees. The success of the silencing is validated by an increase in the level of NPR1. After multiple inoculation with HLB, the modified citrus trees grow normally and experience mild HLB symptoms.

In certain embodiments, disclosed is a CTV viral vector engineered to produce an RNAi inducer targeting CsNPR3 (Cs2g10790). The RNAi inducer may be a dsRNA, siRNA, or miRNA. In a specific embodiment, the RNAi inducer is a dsRNA with a sequence comprising SEQ ID No. 1. The CTV vector may optionally include a sequence that encodes a foreign protein. In a specific embodiment, the foreign protein comprises a defensin.

### Overview

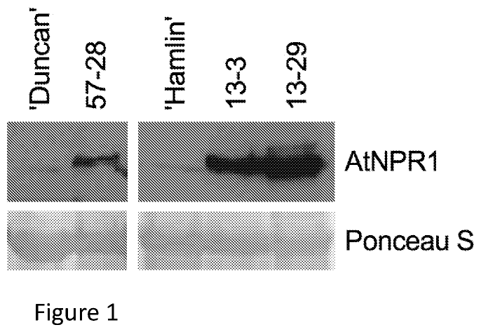

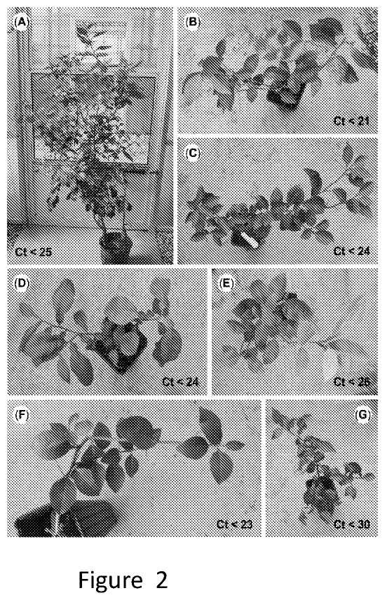

A series of transgenic plants were designed and screened for resistance or tolerance to canker (Zhang et al., 2010) and for HLB resistance or tolerance (Robertson et al., 2018). Two lines in Hamlin sweet orange (13-3 and 13-29) and one line in Duncan grapefruit (57-28) were found to be highly tolerant to HLB. All three lines accumulated significant amounts of the AtNPR1 protein with line 13-29 accumulating the highest level (FIG. 1), revealing a tight correlation between AtNPR1 protein levels and HLB tolerance. These plants were positive for CLas by PCR, but had occasional and minor leaf symptoms and have continued growing normally. Since usefulness of a transgenic citrus line would require it to retain the tolerant phenotype in all the progeny (daughter) plants produced by grafting, the original lines have been propagated into multiple sets of progeny plants four times and with each of the replicates retaining the same level of tolerance. The representative HLB-tolerant transgenic line 13-29 is shown in FIG. 2.

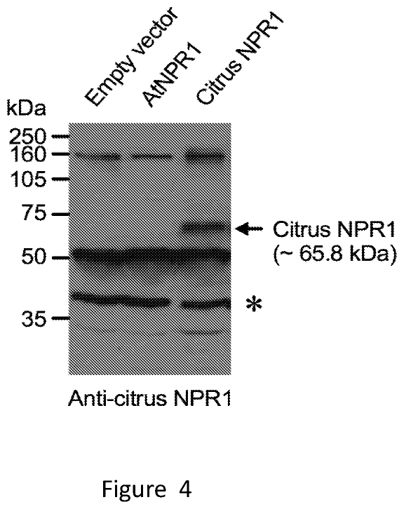

Methods were developed to elevate citrus NPR1 protein levels to achieve HLB tolerance by disrupting negative regulators of the citrus immune system. To identify promising gene targets, the RNAi lines were tested in the greenhouse for HLB responses (Lee et al., 2015). The greenhouse HLB test is time-consuming and requires multiple rounds of testing to confirm resistance or tolerance. Thus, a more straightforward screening method is highly desired to expedite the process. Since the results have shown that AtNPR1 levels tightly correlate with HLB tolerance in the transgenic plants (Robertson et al., 2018), it was reasoned that promising RNAi constructs should simultaneously induce citrus NPR1 protein accumulation and HLB tolerance. Measuring citrus NPR1 protein levels in the RNAi lines would be a much faster approach for identifying tolerance. Unfortunately, the AtNPR1 antibody does not recognize the citrus NPR1 protein. To circumvent this problem, a rabbit polyclonal antibody recognizing the citrus NPR1 was developed. As shown in FIG. 4, this antibody specifically detected the citrus NPR1 protein, but not the AtNPR1 protein, transiently expressed in N. benthamiana leaves. This antibody was used for RNAi line screening.

### CTV-RNAi

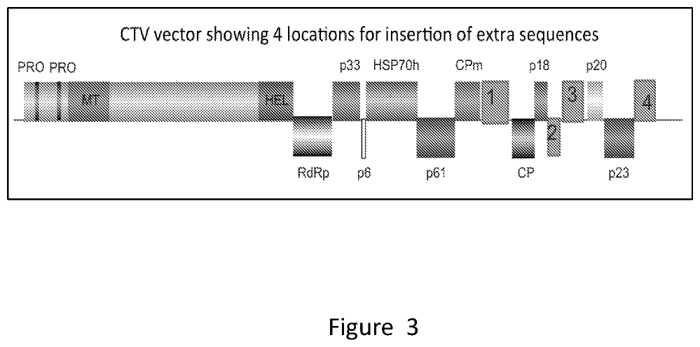

In certain embodiments, a CTV viral vector containing an RNAi inducer targeting is used to create tolerance to HLB in a citrus plant. CTV, a member of the Closteroviridae family, systemically infects citrus. The first effective CTV vector had an insertion of a controller element for producing an extra subgenomic RNA plus an open reading frame of a foreign protein inserted between the two coat protein genes (FIG. 3) (Folimonov et al., 2007). Since then, foreign genes have been shown to be expressed at several locations within the viral genome, allowing modulation of levels of foreign gene expression (El Mohtar, 2011; El Mohtar and Dawson, 2014). Additionally, two or three foreign genes can be expressed simultaneously from the same vector (El Mohtar, 2011; El Mohtar and Dawson, 2014) (FIG. 3). For expression of RNAi inducer sequences to target citrus mRNAs, an extra controller element is not needed, due to the three distinct suppressors of RNA silencing which protects CTV from antiviral silencing machinery of the citrus host (Hajeri et al., 2014). During replication, CTV makes copious amounts of double stranded RNA intermediates, particularly of the subgenomic RNAs (Hilf et al., 1995).

RNA interference (RNAi) is a process by which double-stranded RNA (dsRNA) is used to silence gene expression. While not wanting to be bound by theory, RNAi begins with the cleavage of longer dsRNAs into small interfering RNAs (siRNAs) by an RNaselll-like enzyme, dicer. SiRNAs are dsRNAs that are usually about 19 to 28 nucleotides, or 20 to 25 nucleotides, or 21 to 22 nucleotides in length and often contain 2-nucleotide 3′ overhangs, and 5′ phosphate and 3′ hydroxyl termini One strand of the siRNA is incorporated into a ribonucleoprotein complex known as the RNA-induced silencing complex (RISC). RISC uses this siRNA strand to identify mRNA molecules that are at least partially complementary to the incorporated siRNA strand, and then cleaves these target mRNAs or inhibits their translation. Therefore, the siRNA strand that is incorporated into RISC is known as the guide strand or the antisense strand. The other siRNA strand, known as the passenger strand or the sense strand, is eliminated from the siRNA and is at least partially homologous to the target mRNA. Those of skill in the art will recognize that, in principle, either strand of an siRNA can be incorporated into RISC and function as a guide strand. However, siRNA design (e.g., decreased siRNA duplex stability at the 5′ end of the desired guide strand) can favor incorporation of the desired guide strand into RISC.

The antisense strand of an siRNA is the active guiding agent of the siRNA in that the antisense strand is incorporated into RISC, thus allowing RISC to identify target mRNAs with at least partial complementarity to the antisense siRNA strand for cleavage or translational repression. RISC-related cleavage of mRNAs having a sequence at least partially complementary to the guide strand leads to a decrease in the steady state level of that mRNA and of the corresponding protein encoded by this mRNA. Alternatively, RISC can also decrease expression of the corresponding protein via translational repression without cleavage of the target mRNA.

During replication of CTV, large amounts of double stranded RNA intermediates are produced of the genomic and subgenomic RNAs that are processed into small interfering RNA molecules. The subgenomic RNAs are 3′-coterminal, so the more 3′ sequences are produced multiple times in the longer subgenomic RNAs. Sequences designed to target specific sequences in the plant, pathogen, or pest do not need an extra subgenomic mRNA controller element. Multiple target sequences can be fused together as one larger heterologous sequence.

Single-stranded interfering RNA has been found to effect mRNA silencing, albeit less efficiently than double-stranded RNA. Therefore, embodiments of the present invention also provide for administration of a single-stranded interfering RNA that has a region of at least near-perfect contiguous complementarity with a portion of the target nucleic acid. The single-stranded interfering RNA has a length of about 19 to about 49 nucleotides as for the double-stranded interfering RNA cited above. The single-stranded interfering RNA has a 5′ phosphate or is phosphorylated in situ or in vivo at the 5′ position. The term “5′ phosphorylated” is used to describe, for example, polynucleotides or oligonucleotides having a phosphate group attached via ester linkage to the C5 hydroxyl of the sugar (e.g., ribose, deoxyribose, or an analog of same) at the 5′ end of the polynucleotide or oligonucleotide.

Single-stranded interfering RNAs can be synthesized chemically or by in vitro transcription or expressed endogenously from vectors as described herein in reference to double-stranded interfering RNAs. 5′ Phosphate groups may be added via a kinase, or a 5′ phosphate may be the result of nuclease cleavage of an RNA. A hairpin interfering RNA is a single molecule (e.g., a single oligonucleotide chain) that comprises both the sense and antisense strands of an interfering RNA in a stem-loop or hairpin structure (e.g., a shRNA). For example, shRNAs can be expressed from DNA vectors in which the DNA oligonucleotides encoding a sense interfering RNA strand are linked to the DNA oligonucleotides encoding the reverse complementary antisense interfering RNA strand by a short spacer. If needed for the chosen expression vector, 3′ terminal T's and nucleotides forming restriction sites may be added. The resulting RNA transcript folds back onto itself to form a stem-loop structure.

Many of the embodiments of the disclosure refer to particular methods of inhibiting or disruption of genetic expression. Based on the teachings herein, methods of inhibiting expression include but are not limited to siRNA; ribozyme(s); antibody(ies); antisense/oligonucleotide(s); morpholino oligomers; microRNA; or shRNA that target expression of the target nucleic acid. The disclosure is not to be limited to any of the related methods described. One such method includes siRNA (small interfering/short interfering/silencing RNA). SiRNA most often is involved in the RNA interference pathway where it interferes with the expression of a specific nucleic acid. In addition to its role in the RNA interference pathway, siRNA also act in RNA interference-related pathways, e.g., as an antiviral mechanism or in shaping the chromatin structure of a genome.

Another method by which to inhibit the expression of the target nucleic acid is shRNA. ShRNA (short hairpin or small hairpin RNA) refers to a sequence of RNA that makes a tight hairpin turn and is used to silence gene expression via RNA interference. It uses a vector introduced into cells and a constitutive promoter such as the U6 or H1 promoter to ensure that the shRNA is always expressed. The shRNA hairpin structure is cleaved by cellular machinery into siRNA which is then bound to the RNA-induced silencing complex. This complex binds to and cleaves mRNAs which match the siRNA that is bound to it.

A highly efficient and specific RNAi method called artificial microRNA (amiRNA) is also used to inhibit the expression of the target nucleic acid. The amiRNA method takes advantage of the intrinsic nature of the processing stages of the microRNA biogenesis pathway by replacing the miRNA and miRNA* sequences with artificial sequences while maintaining the double-stranded (dsRNA) stem-loop structure, resulting in production of the designed amiRNA. Similar to shRNA, amiRNA is also introduced into cells using a vector and driven by a constitutive promoter to ensure the amiRNA is always expressed. Examples of amiRNA sequences are provided as SEQ ID NOs: 5-7. An Example of shRNA that may be implemented with the embodiments described herein is provided at SEQ ID NO: 4.

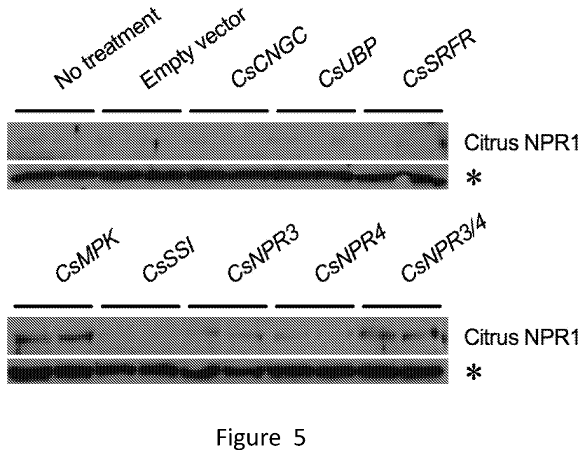

A group of CTV-RNAi constructs targeting potential negative regulators of the citrus immune system was generated. These constructs have been introduced into N. benthamiana and bark flap inoculated into Citrus macrophylla plants. Systemic infection in some of the RNAi lines has been confirmed by ELISA analysis. It was found that the newly developed citrus NPR1 antibody can recognize C. macrophylla NPR1. Thus, NPR1 protein levels were analyzed in the C. macrophylla RNAi lines. Two independent lines were included for each construct to ensure the reproducibility of the observed results. As shown in FIG. 5, while silencing of CsCNGC, CsUBP, CsSRFR, and CsSSI did not cause detectable changes in NPR1 levels, silencing of CsNPR3 and CsNPR4 induced a slight increase. On the other hand, silencing of

CsMPK or CsNPR3 and CsNPR4 together (CsNPR3/4) significantly increased the NPR1 level. Thus, the CsMPK, CsNPR3, CsNPR4, and CsNPR3/4 RNAi inducer sequences have the potential to induce HLB tolerance.

### CRISPR/Cas9

Certain embodiments disclosed utilize CRISPR/Cas9 to create tolerance to HLB in citrus plants. CRISPR/Cas9 is a type II CRISPR/Cas system. The CRISPR/Cas9 system from Streptococcus pyogenes is a versatile tool for RNA guided genome editing in plants including citrus. In this process, a single or duplex short RNA molecule (guide RNA or gRNA) directs Cas9 to the target DNA site for genome editing. The gRNA-Cas9 complex recognizes the target DNA by gRNA-DNA pairing between 5′-end leading sequence of gRNA (called gRNA spacer) and the DNA strand complementary to the protospacer. The target gene sequences for genome editing and genetic modification can be selected using methods known in the art, the online tool CRISPRdirect (https://crispr.dbcls.jp/), and as described elsewhere in this application. In a preferred embodiment, target sequences are identified that include or are proximal to protospacer adjacent motif (PAM). Once identified, the specific sequence can be targeted by synthesizing a pair of target-specific DNA oligonucleotides (gRNA spacer) with appropriate cloning linkers, and phosphorylating, annealing, and ligating the oligonucleotides into the CRISPR/Cas9 binary vector pKSE401 (Xing et al., 2014), or similar CRISPR/Cas9 binary vectors following the described protocols (Xing et al., 2014; Cermak et al., 2017). Two or more gRNA spacers can be assembled into the same CRISPR/Cas9 binary vector via Golden Gate assembly to target more than one gene. The plasmid vector comprising the target-specific oligonucleotides can then be used for transformation of a plant. In specific embodiments, the target gene sequences comprise a negative immune regulator gene.

Gene transfer and genetic transformation methods for introducing engineered gRNA-Cas9 constructs into plant cells include, but are not limited to, protoplast transformation through calcium-, polyethylene glycol (PEG)- or electroporation-mediated uptake of naked DNA (see Paszkowski et al. (1984) EMBO J3:2717-2722; Potrykus et al. (1985) Molec. Gen. Genet. 199:169-177; Fromm et al. (1985) Proc. Nat. Acad. Sci. USA 82:5824-5828; and Shimamoto (1989) Nature 338:274-276) and electroporation of plant tissues (D′Halluin et al. (1992) Plant Cell 4:1495-1505). Additional methods for plant cell transformation include microinjection, silicon carbide mediated DNA uptake (Kaeppler et al. (1990) Plant Cell Reporter 9:415-418), and microprojectile bombardment (see Klein et al. (1988) Proc. Nat. Acad. Sci. USA 85:4305-4309; and Gordon-Kamm et al. (1990) Plant Cell 2:603-618). According to certain embodiments, gene constructs carrying gRNA-Cas9 nuclease or gRNA-Cas9 ribonucleoprotein complexes can be introduced into plant cells by various methods, which include but are not limited to PEG-or electroporation-mediated protoplast transformation, and tissue culture or plant tissue transformation by biolistic bombardment. The gene constructs can also be introduced by the Agrobacterium-mediated transient and stable transformation.

Transformed plant cells which are produced by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker or reporter marker genes (such as GFP or GUS gene) which has been introduced together with the desired nucleotide sequences. Plant regeneration from cultured protoplasts is described in Evans, et al., “Protoplasts Isolation and Culture” in Handbook of Plant Cell Culture, pp. 124-176, Macmillian Publishing Company, New York, 1983; and Binding, Regeneration of Plants, Plant Protoplasts, pp. 21-73, CRC Press, Boca Raton, 1985. Regeneration can also be obtained from plant callus, explants, organs, pollens, embryos or parts thereof. Such regeneration techniques are described generally in Klee et al (1987) Ann. Rev. of Plant Phys. 38:467-486.

### Methods for Creating Tolerance to HLB in Citrus Plants

In certain embodiment, constructs silencing negative immune regulators were created based on CTV infectious cDNA clones that have been inoculated to citrus. In the last few years, many of those constructs have been designed to silence citrus or psyllid genes. For the purpose of developing RNAi targeting plant genes to reduce susceptibility to HLB, either 35S CTV9-47Rp7 or 35S CTV9-47R-136 are used, a modified vector based on 35S CTV9-47R (GenBank accession #AY170468). The unique restriction sites of PacI and StuI are used to introduce the RNAi inducer sequences into the CTV vector. The RNAi inducer sequences are generally 400-600 nt long in an antisense orientation. The StuI and PacI sites are added to each of the forward and reverse primer sets, respectively. Ligation and transformations are done according to standard molecular procedures established for CTV cloning known to ones skilled in the arts. All CTV vector plasmid clones are checked for the right insert size by restriction digestion with PstI and StuI followed by sequencing.

In certain embodiments, an efficient Agro-inoculation procedure for CTV vectors into N. benthamiana modified was established from Ambros et al. (2011). Recombinant virions of CTV vectors isolated from systemic leaves of N. benthamiana are concentrated by centrifugation in a step gradient followed by a cushion gradient in SW28 and SW41 rotors, respectively (Garnsey and Henderson, 1982). Inoculation of citrus plants is done by bark flap inoculation into 1-1.5-year-old C. macrophylla seedlings (Robertson et al., 2005). Establishment of systemic infection in the plants is validated by ELISA analysis. Silencing of the target genes is assessed by qPCR analysis.

As CTV is phloem limited, phloem-associated tissues are the focus. Bark tissues are used as the example to describe the method. Other tissues are similarly assayed. About 100 mg bark tissues is collected from each RNAi line and snap frozen in liquid N₂. Total protein is extracted, separated by SDS-PAGE, transferred onto nitrocellulose, and probed with the citrus NPR1 antibody as in FIG. 5. The antibody-bound proteins are detected using a horseradish peroxidase-conjugated anti-rabbit secondary antibody followed by chemiluminescence detection and quantification.

### EXAMPLES

### Example 1

This embodiment is based on the transgenic results, increasing NPR1 levels is expected to provide tolerance that alleviate or suppress HLB symptoms but not reduce CLas titers in the plants. On the other hand, spinach defensins have been used to reduce the pathogen titers. It is reasonable to expect that NPR1-mediated host tolerance and defensin-mediate pathogen reduction can act synergistically in battling against the HLB disease. The promising RNAi inducer sequences will be inserted between the 3′-most gene (p23) and the non-translated 3′ sequence (location 4 in FIG. 3) of the CTV-defensin constructs that Southern Gardens Citrus has been testing in the field in the past 10 years. The unique restriction sites of PacI and StuI are used to introduce the RNAi inducer sequences into the CTV vector. The RNAi inducer sequences are generally 400-600 nt long in an antisense orientation. The StuI and PacI sites are added to each of the forward and reverse primer sets, respectively. Ligation and transformations are done according to standard molecular procedures established for CTV cloning known to ones skilled in the arts.

In certain embodiments, an efficient Agro-inoculation procedure for CTV vectors into N. benthamiana modified was established from Ambros et al. (2011). Recombinant virions of CTV vectors isolated from systemic leaves of N. benthamiana are concentrated by centrifugation in a step gradient followed by a cushion gradient in SW28 and SW41 rotors, respectively (Garnsey and Henderson, 1982). Inoculation of citrus plants is done by bark flap inoculation into 1-1.5-year-old C. macrophylla seedlings (Robertson et al., 2005). Establishment of systemic infection in the plants is validated by ELISA analysis. Silencing of the target genes is assessed by qPCR analysis.

C. macrophylla is tolerant to HLB and cannot be directly screened for tolerance. After systemic infection is established in C. macrophylla and validated with ELISA results, the CTV-defensin-RNAi constructs are graft-transmitted into sweet orange or grapefruit seedlings for HLB phenotype screening. Both uninfected and HLB-infected seedlings are included in the experiment. ELISA is employed to monitor the establishment of systemic infection of CTV in the trees. After systemic CTV infection is established, the citrus trees are molecularly characterized. Silencing of the negative regulators and expression of the defensin gene is confirmed by qPCR, and the RNAi-induced increase in NPR1 levels is verified by western blot analysis using the citrus NPR1 antibody.

For the HLB-infected trees, the therapeutic effect of CTV-defensin-RNAi constructs is assessed via monitoring HLB symptom development and CLas titer reduction. For preventative effect, the evaluation is the same as used to identify tolerance in the transgenic citrus (Robertson et al., 2018). The citrus trees are inoculated with HLB by incubating them in a containment plant growth room with CLas-infected psyllids, usually for 2-4 weeks. Effective silencing of the promising negative regulators increases the citrus NPR1 levels and provide resistant or tolerant phenotypes.

### Example 2

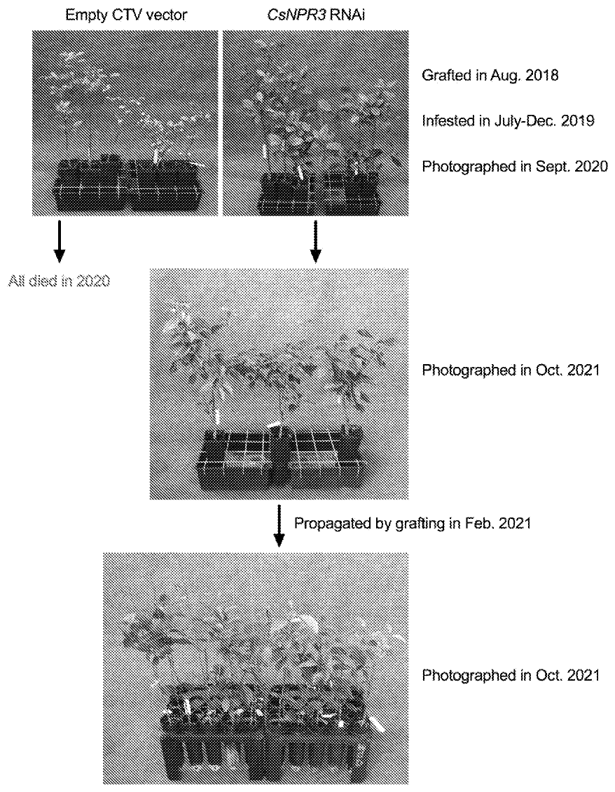

In certain embodiments, a large number of C. macrophylla trees are inoculated with the CsNPR3/4 CTV-defensin-RNAi. After systemic CTV infection is established and validated with ELISA, stem or bark pieces from the C. macrophylla trees are grafted onto both HLB-infected and uninfected trees in the field to evaluate the therapeutic and preventative effects of the constructs, respectively. Trees are arranged in the field based on a randomized complete block or a completely randomized design. These trees are evaluated for HLB symptoms and CLas titers after two years. FIG. 6 provides photographs of a Huanglongbing (HLB) test result of control plants (left) and CsNPR3 RNAi lines (right). The control plants were infected with the CTV empty vector. CTV and CTV-RNAi constructs were graft-transmitted into sweet orange ‘Pineapple’ seedlings. After systemic CTV infection was confirmed with ELISA, the citrus trees were inoculated in a containment growth room with CLas-infected psyllids for 2-4 weeks. The plants were all successfully infested. The control plants carrying the CTV empty vector exhibited severe HLB symptoms, whereas the CsNPR3 RNAi lines showed no or very mild HLB symptoms. This result indicates that CsNPR3 RNAi provided robust tolerance to the HLB disease.

While the invention is susceptible to various modifications and alternative forms, specific embodiment thereof has been shown by way of example in the drawings and will be described in detail below. It should be understood, however that it is not intended to limit the invention to the particular forms disclosed, but on the contrary, the invention is to cover all modifications, equivalents, and alternatives falling within the spirit and the scope of the invention. All documents, patents, journal articles and other materials cited herein are incorporated in their entirety to the extent not inconsistent with the teachings herein.

### REFERENCES

Ambros, S., El-Mohtar, C., Ruiz-Ruiz, S., Peña, L., Guerri, J., Dawson, W. O., and Moreno, P. (2011). Agroinoculation of Citrus tristeza virus causes systemic infection and symptoms in the presumed non-host Nicotiana benthamiana Mol. Plant Microbe Interact 24, pp. 1119-1131
Basu, S., Sharma, V. K., Bhattacharyya, D., and Chakraborty, S. (2014). An overview of antiviral RNA silencing in plant: biogenesis, host-virus interaction and potential application. In Gaur R. K., and Sharma, P. (ed.). Springer, India
Blaustein, R. A., Lorca, G. L., and Teplitski, M. (2017). Challenges for managing Candidatus Liberibacter spp. (Huanglongbing disease pathogen): current control measures and future directions. Phytopathol 108,424-435.
Ding, S. W., and Lu, R. (2011). Virus-derived siRNAs and piRNAs in immunity and pathogenesis. Curr Opin Virol 1, 533-544.
El Mohtar, C. (2011). Exploring Citrus tristeza virus-based limits for foreign gene expression. University of Florida.
El-Mohtar, C., and Dawson, W. O. (2014). Exploring the limits of vector construction based on Citrus tristeza virus. Virology 448, 274-283.
FL DACS (2020). Florida citrus stattistics.
Folimonov, A. S., Folimonova, S. Y., Bar-Joseph, M., and Dawson, W. O. (2007). A stable RNA virus-based vector for citrus trees. Virology 368, 205-216.Graham, J., and Morgan, K. (2017). Why bicarbonates matter for HLB management. Citrus Industry 98, 16-21.
Graham, J., Gottwald, T., and Setamou, M. (2020). Status of Huanglongbing (HLB) outbreaks in Florida, California and Texas. Tropical Plant Pathol (https://doi.org/10.1007/s40858-020-00335-y).
Garnsey, S. M., and Henderson, C. T. (1982). Extraction, centrifugation, and assay techniques for purification of intact citrus tristeza virus. Workshop on Plant Virus Detection, Agric. Exp. Stn., University of Puerto, Rico, Rio Piedras, Mar. 29-Apr. 2, 1982, p106-112.
Hajeri, S., Killiny, N., El-Mohtar, C., Dawson, W. O., and Gowda, S. (2014). Citrus tristeza virus-based RNAi in citrus plants induces gene silencing in Diaphorina citri, a phloem-sap sucking insect vector of citrus greening disease (Huanglongbing). J Biotechnol 176, 42-49.
Hilf, M. E., Karasev, A. V., Pappu, H. R., Gumpf, D. J., Niblett, C. L., and Garnsey, S. M. (1995). Characterization of citrus tristeza virus subgenomic RNAs in infected tissue. Virology 208, 576-582.
Lee, J. A., Halbert, S. E., Dawson, W. O., Robertson, C. J., Keesling, J. E., and Singer, B. H. (2015). Asymptomatic spread of huanglongbing and implications for disease control. Proc Natl Acad Sci USA 112, 7605-7610.
Kumagai, L., LeVesque, C. S., Blomquist, C. L., Madishetty, K., Guo, Y., Woods, P. W., Rooney-Latham, S., Rascoc, J., Gallindo, T., Schanbel, D., and Polek, M. (2013). First report of Candidatus Liberibacter asiaticus associated with citrus Huanglongbing in California. Plant Disease 92, 283.
MacDiarmid, R. (2005). RNA silencing in productive virus infections. Annu Rev Phytopathol 43, 523-544.
McRoberts, N., Figuera, S. G., Olkowski, S., McGuire, B., Luo, W., Posny, D., and Gottwald, T. (2019). Using models to provide rapid programme support for California's efforts to suppress Huanglongbing disease of citrus. Philos T R Soc B 374, 20180281
Robertson, C. J., Garnsey, S. M., Satyanarayana, T., Folimonova, S., and Dawson, W. O. (2005). Efficient infection of citrus plants with different cloned constructs of Citrus tristeza virus amplified in Nictiana benthamiana protoplasts. Proc. 16th Conf. IOCV. IOCV, Riverside, CA, p187-195.
Zhang, X., Francis, M. I., Dawson, W. O., Graham, J. H., Orbović, V., Triplett, E. W., and Mou, Z. (2010). Overexpression of the Arabidopsis NPR1 gene in citrus increases resistance to citrus canker. Eur J Plant Pathol 128, 91-100.

1. A method for activating systemic acquired resistance (SAR) in a citrus plant, the method comprising introducing into a citrus plant cell or tissue of the citrus plant a non-naturally occurring Citrus tristeza virus (CTV) viral vector engineered to produce an RNA interfering (RNAi) inducer sequence targeting negative immune regulators such as CsNPR3 (Cs2g10790), wherein the RNAi inducer sequence silences CsNPR3 to increase SAR in the citrus tree.

2. The method of claim 1, wherein said SAR provides tolerance to Huanglongbing (HLB).

3. The method of claim 1, wherein the CTV vector comprises a controller element for producing a RNAi inducer sequence, and wherein the CTV vector may optionally include a sequence that encodes a foreign protein.

4. (canceled)

5. The method of claim 4 3, wherein the foreign protein comprises a defensin.

6. The method of claim 1, wherein the RNAi inducer sequence is cloned from citrus complementary DNA (cDNA).

7. The method of claim 1, wherein the RNAi inducer sequence expresses dsRNA, shRNA, or amiRNA targeting CsNPR3 (Cs2g10790).

8. The method of claim 7, wherein the sequence expressing dsRNA comprises SEQ ID NO. 1.

9. The method of claim 7, wherein the sequence expressing shRNA comprises SEQ ID NO. 4.

10. The method of claim 7, wherein the sequence expressing amiRNA comprises SEQ ID NO. 5, SEQ ID NO. 6, or SEQ ID NO. 7.

11. The method of claim 1, wherein the CTV viral vectors are introduced into the citrus plant by bark flap inoculation.

12. (canceled)

13. A method for activating SAR in a citrus plant, the method comprising introducing into a citrus plant cells a non-naturally occurring gene editing system comprising (i) a nuclease encoding a Clustered Regularly Interspaced Short Palindromic Repeats (CRISPR)-associated (Cas) protein and (ii) a gRNA that targets CsNPR3 (Cs2g10790), wherein the gene editing system disrupts or alters CsNPR3 expression or function.

14. The method of claim 13, wherein said sequence encoding a Type-II CRISPR-associated nuclease is operably linked to a terminator sequence functional in a plant cell.

15. The method of claim 13, wherein said type II CRISPR-associated nuclease is Cas9.

16. (canceled)

17. The method of claim 13, wherein the gRNA spacer sequence comprises SEQ ID NO. 2 and/or SEQ ID NO. 3.

18. (canceled)

19. The method of claim 13, wherein disrupting or altering CsNPR3 expression or function activates SAR in the citrus plant.

20. The method of claim 19, wherein said SAR provides tolerance to HLB.

21. The method of claim 1, wherein said citrus plant is a sweet orange, bitter orange, blood orange, naval orange, grapefruit, pomelo, citron, lemon, lime, Clementine, mandarin, tangerine, tangelo, tangor, trifoliate orange, trifoliate orange hybrid, kumquat plant.

22. A CTV viral vector engineered to produce an RNAi inducer targeting CsNPR3 (Cs2g10790), wherein the CTV vector comprises a controller element for producing a RNAi inducer sequence, and wherein the CTV vector may optionally include a sequence that encodes a foreign protein.

23. (canceled)

24. (canceled)

25. The vector of claim 22, wherein the foreign protein comprises a defensin.

26. (canceled)

27. The vector of claim 22, wherein the RNAi inducer sequence expresses dsRNA, shRNA, or amiRNA targeting CsNPR3 (Cs2g10790), wherein the sequence expressing dsRNA comprises SEQ ID NO. 1, wherein the sequence expressing shRNA comprises SEQ ID NO. 4, and wherein the sequence expressing amiRNA comprises SEQ ID NO. 5, SEQ ID NO. 6, or SEQ ID NO. 7.

28. (canceled)

29. (canceled)

30. (canceled)

31. (canceled)

32. (canceled)
